(19) United States
(12) Patent Application Publication
(10) Pub. No.: US 2021/0030259 A1
(43) Pub. Date: Feb. 4, 2021

(54) Surgical Airflow Device

(71) Applicant: Medabotics, Inc., Knoxville, TN (US)

(72) Inventor: Xiaolong Liu, College Park, MD (US)

(21) Appl. No.: 16/530,043

(22) Filed: Aug. 2, 2019

(51) Int. Cl. A61B 1/00; A61B 1/12; A61B 1/015; F04B 37/10; F04B 43/04

(52) U.S. Cl. CPC A61B 1/00091; A61B 1/126; A61B 1/313; F04B 37/10; F04B 43/046; A61B 1/015

(57) ABSTRACT

An airflow device projects a flow of air proximate to a field of view of a camera. The airflow device includes: a housing shaped to fit over an end of a camera lens; one or more air pumps located within the housing, the one or more air pumps receiving air through one or more air inlets; and one or more air outlets formed in the housing proximate the camera lens. The one or more air outlets are oriented to project air from the one or more air pumps across a field of view of the camera lens.

### FIELD

This disclosure relates to the field of surgical devices. More particularly, this disclosure relates to a device for cleaning a lens of a surgical instrument used for minimally invasive surgery.

### BACKGROUND

Clear visualization of the surgical field in minimally invasive surgery (MIS) is vitally important for the surgeon's operation efficiency and the patient's safety. Surgical scope cameras have been developed with various dimensions, flexibility, and controllability to reach inside human bodies for various MIS procedures, such as laparoscopic, endoscopic, bronchoscopic, arthroscopic, and thoracoscopic. Surgeons manipulate surgical instruments to carry out surgical procedures solely based on the visual guidance of these scope cameras. Uninterrupted and clear visual guidance is critical to the success of all these surgeries.

However, clarity of a visual field of a surgical scope camera 1 can easily be impaired by contaminated lenses due to vapor condensation, particle debris, rinsing fluid, and body fluid 2; or accumulated smoke 3 caused by electrocautery during surgery as shown in FIG. 1. According to clinical studies, more than 37% of laparoscopic surgery time is done with impaired rigid laparoscopes due to contamination of camera lenses that is considered troublesome by 68% surgeons. About 3% extra time during laparoscopic surgery is required to externally clean the blinded camera lenses, which increases the procedure length and surgery cost. More seriously, the distraction may affect a surgeon's judgment, and cause patient injury. A visually impaired rigid laparoscope could be removed and cleaned externally. However, the situation for flexible scope cameras which reach deep inside body cavity or for remotely controlled robotic surgical devices could be catastrophic for both patients and surgeons.

Surgical smoke, also known as cautery smoke or electrosurgery smoke, is produced by interaction of mechanical and heat producing instruments with tissue, such as dissection and homeostasis tools. Generated smoke is a gaseous byproduct of the disruption and vaporization of tissue, protein, and fat. Fogging or smoke can deteriorate vision of a camera used during a procedure such as by blocking a field of view of the camera.

What is needed, therefore, is a modular surgical lens cleaning device that functions to evacuate smoke plumes to maintain visual clarity of robotic surgical cameras inside a body cavity.

### SUMMARY

The above and other needs are met by an airflow device for projecting a flow of air proximate to a field of view of a camera. In a first aspect, the airflow device includes: a housing shaped to fit over an end of a camera lens; one or more air pumps located within the housing, the one or more air pumps receiving air through one or more air inlets; and one or more air outlets formed in the housing proximate the camera lens. The one or more air outlets are oriented to project air from the one or more air pumps across a field of view of the camera lens.

In one embodiment, the one or more air outlets include: one or more projecting outlets oriented to project air from the one or more air pumps away from an end of the housing and substantially parallel to a field of view of the camera and one or more lens outlets oriented to projected air from the one or more air pumps perpendicular to and across a field of view of the camera.

In another embodiment, the the one or more air pumps are piezoelectric air pumps. In yet another embodiment, the one or more air pumps are piezoelectric air pumps are arranged in an array around the housing of the airflow device. In one embodiment, the piezoelectric air pumps are arranged in an array and are located within walls of the housing of the airflow device.

In another embodiment, the one or more air pumps further comprising at least one check valve in fluid communication with an interior of the one or more air pumps.

In yet another embodiment, the housing includes a lens cover attached to the housing and a camera chamber located between the lens cover and the camera when the airflow device is installed on the camera.

In a second aspect, an airflow device includes: a housing shaped to fit over an end of a camera lens; one or more air pumps located within the housing, the one or more air pumps receiving air through one or more air inlets, the one or more air pumps comprising piezoelectric air pumps; one or more air outlets formed in the housing proximate the camera lens. The one or more air outlets are oriented to project air from the one or more air pumps across a field of view of the camera lens.

In one embodiment, the one or more air pumps are piezoelectric air pumps are arranged in an array around the housing of the airflow device.

In another embodiment, the one or more air outlets include: one or more projecting outlets oriented to project air from the one or more air pumps away from an end of the housing and substantially parallel to a field of view of the camera and one or more lens outlets oriented to projected air from the one or more air pumps perpendicular to and across a field of view of the camera.

In yet another embodiment, the piezoelectric air pumps are arranged in an array and are located within walls of the housing of the airflow device.

In a third aspect, an airflow device includes: a housing shaped to fit over an end of a camera lens; one or more air pumps located within the housing, the one or more air pumps receiving air through one or more air inlets; one or more air outlets formed in the housing proximate the camera lens, the one or more air outlets are oriented to project air from the one or more air pumps across a field of view of the camera lens, the one or more air outlets including: one or more projecting outlets oriented to project air from the one or more air pumps away from an end of the housing and substantially parallel to a field of view of the camera and one or more lens outlets oriented to projected air from the one or more air pumps perpendicular to and across a field of view of the camera.

### BRIEF DESCRIPTION OF THE DRAWINGS

Further features, aspects, and advantages of the present disclosure will become better understood by reference to the following detailed description, appended claims, and accompanying figures, wherein elements are not to scale so as to more clearly show the details, wherein like reference numbers indicate like elements throughout the several views, and wherein:

FIG. 1 shows an airflow device installed on a camera according to one embodiment of the present disclosure;

FIG. 2 shows an exploded view of an airflow device according to one embodiment of the present disclosure;

FIG. 3 shows an airflow device according to one embodiment of the present disclosure;

FIG. 4 shows a cross-sectional side view of a housing of an airflow device according to one embodiment of the present disclosure;

FIG. 5 shows a view of an airflow device and one or more air pumps according to one embodiment of the present disclosure;

FIG. 6A shows a side view of air pumps of an airflow device according to one embodiment of the present disclosure;

FIG. 6B shows a cross-sectional side view of an air pump of an airflow device according to one embodiment of the present disclosure;

FIG. 6C shows a front view of a diaphragm of an air pump according to one embodiment of the present disclosure;

FIG. 7 shows a simulated chart of a CO2 insufflated abdominal cavity with a pressure of 1.6 kPa and a temperature of 37 C;

FIG. 8A shows a bottom perspective view of an airflow device according to one embodiment of the present disclosure;

FIG. 8B shows a side view of an airflow device according to one embodiment of the present disclosure;

FIG. 8C shows a cross-sectional side view of an airflow device according to one embodiment of the present disclosure;

FIG. 9A shows a schematic view of airflow from one or more projecting outlets of an airflow device according to one embodiment of the present disclosure; and

FIG. 9B shows a schematic view of airflow from a lens outlet of an airflow device according to one embodiment of the present disclosure.

### DETAILED DESCRIPTION

Various terms used herein are intended to have particular meanings. Some of these terms are defined below for the purpose of clarity. The definitions given below are meant to cover all forms of the words being defined (e.g., singular, plural, present tense, past tense). If the definition of any term below diverges from the commonly understood and/or dictionary definition of such term, the definitions below control.

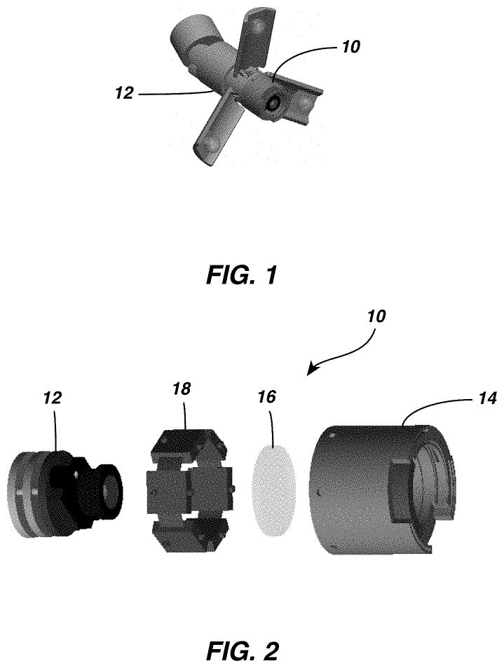

FIG. 1 shows a basic embodiment of an airflow device 10 adapted to be installed on a camera 12, such as an arthroscopic camera inserted into a cavity during a surgical procedure. The airflow device 10 advantageously generates a sufficient airflow around a lens of a camera to prevent obstruction of a field of view of the camera 12 during a surgical procedure. The airflow device 10 is preferably modular and configured such that the airflow device 10 may be installed over an existing camera or lens of a camera to maintain a clear field of view of the camera.

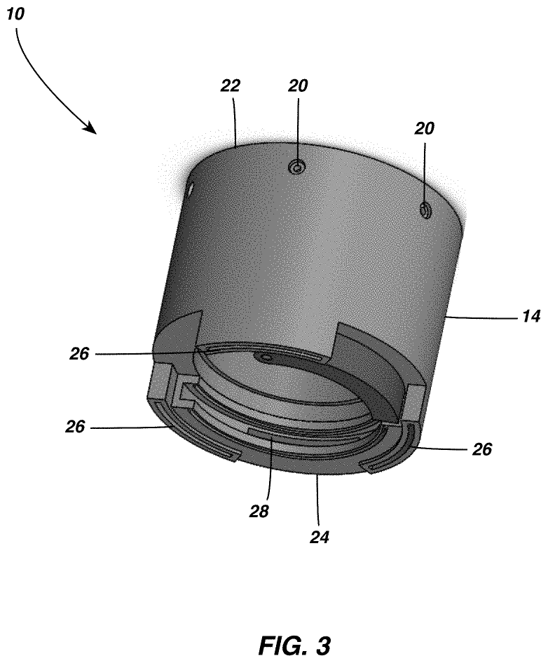

Referring to the exploded view of FIG. 2, the airflow device 10 includes a housing 14 that is shaped to fit on the camera 12. The airflow device 10 further includes a lens cover 16 that is shaped to fit over a lens of the camera 12 and that preferably fits within the housing 14 as described in greater detail below. One or more air pumps 18 are further provided for generating an airflow to prevent obstruction of the field of view of the camera 12 according to embodiments described herein.

The housing 14 preferably has a circular cross-sectional area and is further preferably cylindrical in shape having opposing open first and second ends. The housing 14 preferably contains the one or more air pumps 18 and is configured to direct one or more currents of air generated by the one or more air pumps 18 through the housing 14 and out of an end of the housing 14 that is distal from the camera 12 when the housing 14 is installed on the camera 12.

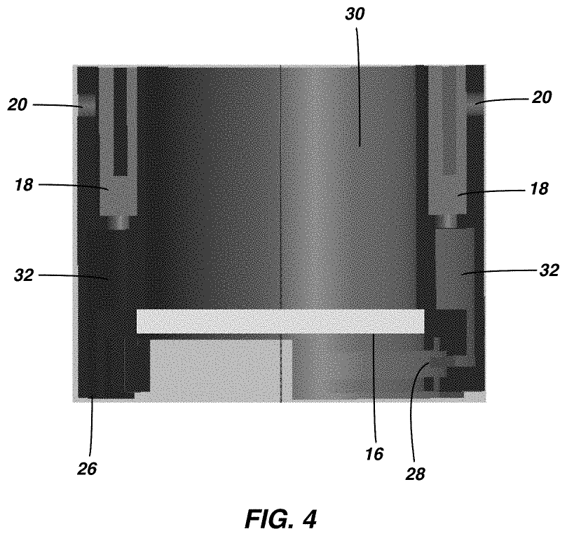

Referring now to FIG. 3, the housing 14 preferably includes a plurality of air intake ports 20 formed around an outer surface of the housing 14 for receiving intake air of the one or more air pumps 18. The plurality of air intake ports 20 are preferably located towards a first end 22 of the housing 14 that is adjacent to the camera 12. The housing 14 further includes a plurality of air outlets located on a second end 24 of the housing 14 that are distal from the camera 12. The plurality of air outlets include one or more projecting outlets 26 that are preferably formed as slots and are oriented to discharge air current in a direction that is outward from the second end 24 of the housing 14 and parallel to an elongate axis of the housing 14. The plurality of air outlets further include at least one lens outlet 28 that is also preferably formed as at least one slot and oriented to discharge air current in a direction that is perpendicular to the elongate axis of the housing 14 and across the camera 12, such as across the lens cover 16 of the airflow device 10.

The one or more projecting outlets 26 are preferably formed around a circumference of the second end 24 of the housing 14 such that the one or more projecting outlets 26 do not obscure a field of view of the camera 12. The at least one lens outlet 28 is preferably formed as a slot formed through an inner wall of the housing 14 such that air flowing out of the lens outlet 28 flows perpendicular across the housing 14.

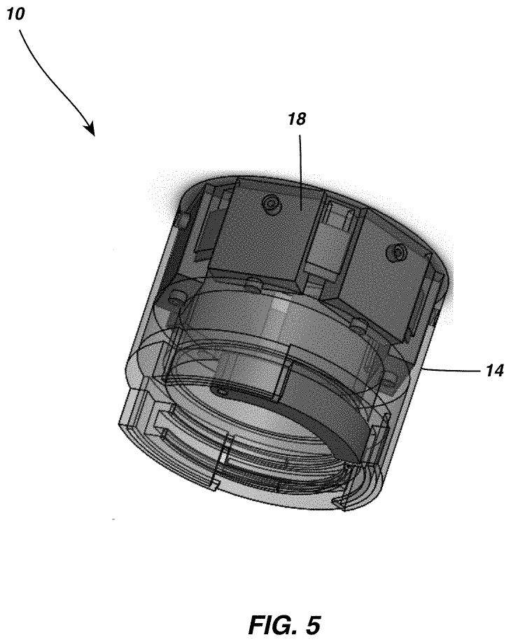

FIG. 4 shows a cross-sectional side view of the airflow device 10. The housing 14 defines a camera chamber 30 shaped to receive at least a portion of the camera 12 to prevent a lens of the camera 14 from becoming contaminated with any debris or being obscured by smoke. The camera chamber 30 preferably forms a substantially air-tight seal between the camera 12, inner walls of the housing 14 and the lens cover 16. The one or more air pumps 18 are preferably located within walls of the housing 14 and in fluid communication with the plurality of air intake ports 20. An outlet of the one or more air pumps 18 is in fluid communication with an air chamber 32 formed within the housing 14, as shown in FIG. 4. Currents of air generated by the one or more air pumps 18 flow through the air chamber 32 and are emitted from the one or more projecting outlets 26 and at least one lens outlet 28.

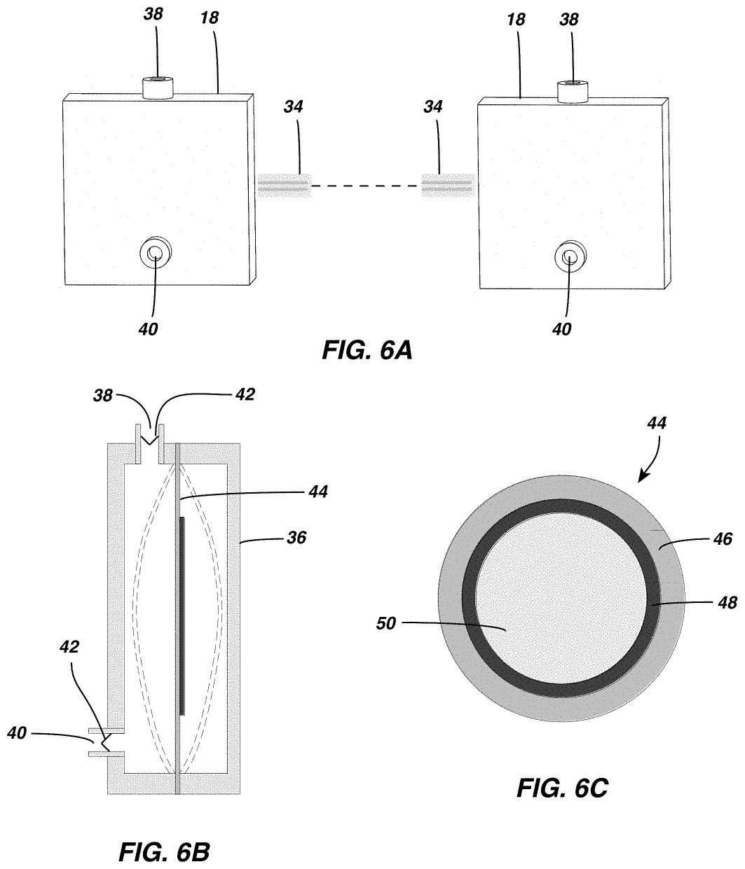

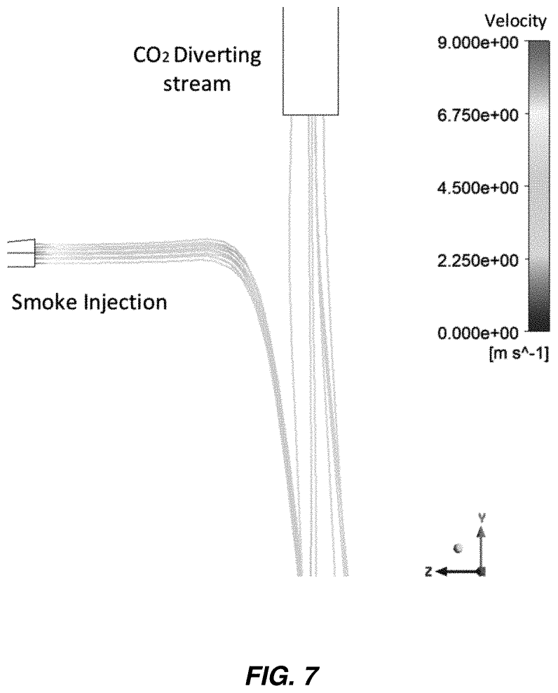

Referring now to FIG. 5, the one or more air pumps 18 are preferably piezoelectric (PZT) air pumps that feature a simple structure, are small in dimension, have high energy efficiency and low power consumption, emit low noise, and have a long operating life. PZT air pumps formed according to embodiments described herein may circulate air within body cavities without affecting an internal cavity pressure. Suitable PZT air pumps are preferably provided having a diameter of approximately 20 mm and a thickness of approximately 1.85 mm and are capable of generating an airflow of approximately 1.62 L/min. According to preferable embodiments herein, a plurality of PZT air pumps are arranged in an array around and within a circumference of the housing 14. Referring to FIG. 6A, each of the one or more air pumps 18 preferably has dimensions of from about 4 mm to about 8 mm in length and from about 2 mm to about 3 mm in thickness. Each of the one or more air pumps 18 is preferably in electronic communication with other of the one or more air pumps 18 via a connector 34, such as a flexible PCB.

Referring now to FIG. 6B, each of the one or more air pumps 18 is preferably a PZT air pump having a pump case 36 including a pump outlet 38 and a pump inlet 40, the pump outlet 38 and pump inlet 40 in fluid communication with an interior of the pump case 36. The pump outlet 38 and pump inlet 40 both preferably include at least one check valve 42 to prevent backflow of any air or fluid into the pump case 36 during operation of the air pump 18. A diaphragm 44 (FIG. 6C) is preferably located within the pump case 36. The diaphragm 44 includes a metal plate 46, a piezoelectric film 48 located thereon. The diaphragm 44 further includes an electrode 50 formed on the diaphragm 44. The diaphragm 44 is actuated within the pump case 36 by the electrode 50 and the metal plate 46 such that the diaphragm 44 is displaced, as shown in FIG. 6B such that air is drawn in through the pump inlet 40 and expelled through the pump outlet 38. The at least one check valve 42 controls a direction of flow of air such that air is emitted from the pump outlet 38. A velocity of air flow generated by the one or more air pumps 18 may be calculated, for example, by an excitation frequency a) and vibration displacement amplitude of the diaphragm 44, and governed by motion for a damped system as Mü+C\dot{u}+Ku=F, where M is a structural mass matrix, K is a structural stiffness matrix, C is a structural damping matrix, and F is an applied load vector. Dimensions and a configuration of the one or more air pumps 18 may vary depending on desired air flow speed for diverting contaminants away from the camera 10.

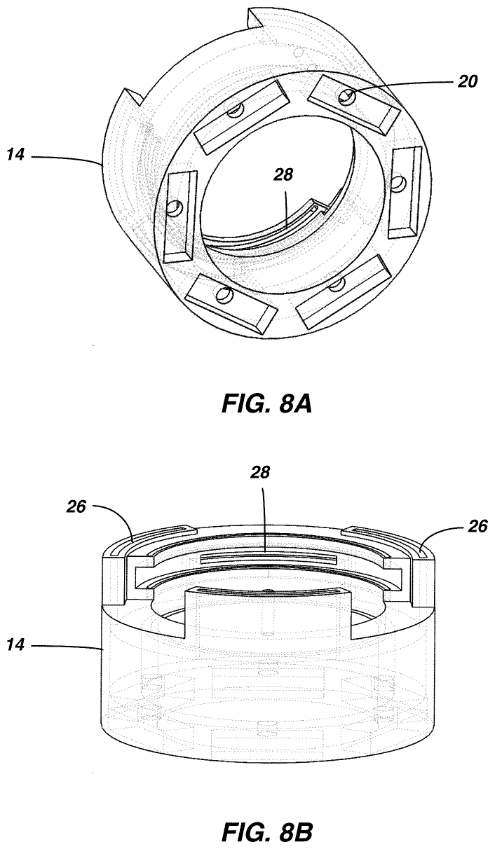

In one exemplary embodiment, a minimum flow speed for diverting contaminants such as smoke, water, and bloodstreams is determined. As shown in FIG. 7, in one example a CO2 insufflated abdominal cavity is simulated with a pressure of 1.6 kPa and a temperature of 37 C. Simulated smoke, water, and blood streams are injected towards the Co2 stream, blow from a linear nozzle with a 1 mm exit gap. As shown in FIG. 7, a minimum CO2 velocity for diverting approaching streams is found to be 3 m/s in various contamination scenarios.

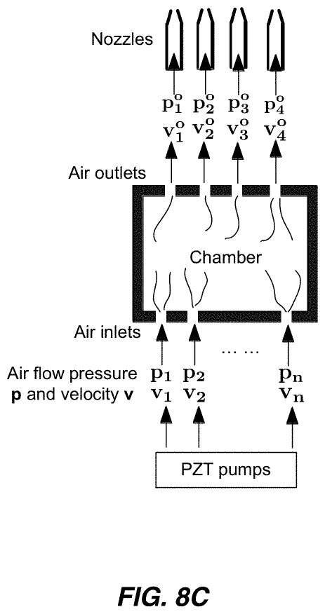

Air flows generated by the one or more air pumps 18, and preferably the PZT air pumps, preferably merge within the housing 14 and are emitted from the projecting outlets 26 and at least one lens outlet 28 to form a shield of air and a blade of air protecting a clarity of the lens of the camera 12, as shown in FIGS. 8A-8C. Inlet and outlet flow pressures and velocities may be defined as pᵢ, pᵢ^{o} and vᵢ, vⱼ^{o} (i=1, . . . , n, j=1, 2, 3, 4, n denotes a number of PZT pumps). Given a prescribed air flow distribution pⱼ^{o} and vⱼ^{o} on the projecting outlets 26, a structure of the housing 14 is optimized such that intake air flows meet output airflow requirements. To calculate an optimized structure for inner chambers of the housing 14, governing equations for compressible air flow are represented as

$$\frac{\partial ρ}{\partial t} = - ρ ∇ \cdot v , \dot{v} = F - \frac{∇ p}{ρ} , and ρ \dot{h} = \dot{p} + ∇ \cdot ( k ∇ T ) + Φ ,$$

where h is enthalpy; k is air thermal conductivity; T is temperature; and Φ is a function of viscous dissipation.

Embodiments of the airflow device 10 advantageously generate one or more airflows in front of the camera 12 to prevent contamination of the camera 12. The airflow device 10 provides a prevention area that is preferably hemispherical in shape and centered around a field of view of the camera 12. Any contamination approaching the camera 12 is deflected to maintain a clear field of view of the camera 12. The projecting outlets 26 and at least one lens outlet 28 are preferably arranged to substantially uniformly project air around the camera 12. Further, the at least one lens outlet 28 projects a blade of air across the lens cover 16 for peeling off any contaminants that may have adhered to the lens cover 16 during a procedure.

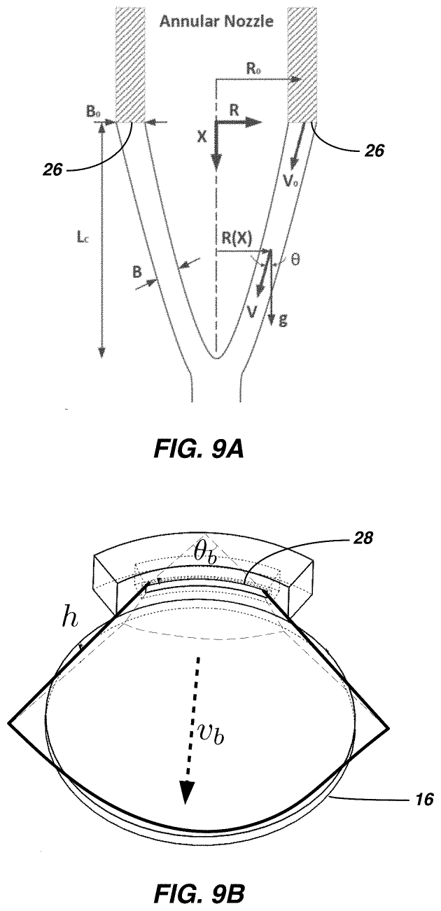

A profile of airflow generated by the projecting outlets 26 is governed by parameters of the projecting outlets 26: an outlet gap B₀, outlet air velocity V₀, nozzle radius R₀, and characterized by a radius of the profile R=fƒ(x, R₀, B₀, p, σ, ΔP), which is a function of air flow thickness B, gravity g, surface tension σ, a pressure difference on two sides of the air flow ΔP, and nozzle parameters , as shown in FIG. 9A. By assuming that the projecting outlets 26 are thin walled (R₀/B₀>>1), an air shield profile R is governed by a continuity equation BRV cos θ=B₀R₀V₀cos θ₀, an axial momentum equation

$$\ddot{X} = g - \frac{2 σ}{ρ B R} sin θ cos θ - \frac{2 σ}{ρ B {R}_{c}} ,$$

and, radial momentum equation

$$\ddot{R} = \frac{2 σ}{ρ B R} {cos}^{2} θ - \frac{2 σ}{ρ B {R}_{c}} cos θ$$

in cylindrical coordinates, where X is a vertical coordinate, Rc is an air flow's radius of curvature in a vertical plane. It has been found that because a near-vertical air flow is created, an angle θ is small except near a closure area. Inner and outer pressures a flow are assumed as equal. Further, the flow radius R and radius of curvature Rc are assumed to be positive.

The at least one lens outlet 28 preferably projects a flat-fan air flow onto the cover lens 16 of the airflow device 10, as shown in FIG. 9B. A design and analysis of the at least one lens outlet 28 may benefit from a theoretical atomization model for flat-fan spray nozzles. A flat-fan fluid flow jetting from a nozzle includes three phases: near field high velocity continuous flow; middle field instable turbulent flow; and far field droplet flow. Performance may be based on parameters including velocity v_{b}, thickness h, and fan angle θ_{b}.

Embodiments of the airflow device 10 described herein prevent or reduce the accumulation of debris on the camera 12, such as during a surgical procedure in which the camera 12 is inserted into a cavity. The projecting outlets 26 and at least one lens outlet 28 act to create a “shield” of air projecting outward from the camera 12 while further creating a “blade” of air that moves across a field of view of the camera 12. Together, airflow from the projecting outlets 26 and at least one lens outlet 28 cooperate to prevent accumulation of debris or to prevent debris from contacting the cover lens 16 or any portion of the camera 12 to ensure that a field of view of the camera 12 remains unobstructed.

Additional advantages of the airflow device 10 include integration of the airflow device 10 with a wiper or other device that contacts the cover lens 16 or the camera 12 to clear debris from a field of view of the camera 12. For example, the at least one lens outlet 28 may acts to clear debris from the camera 12 after a wiper or other device clears a lens of the camera 12. Further, the airflow device 10 may act to cool components of a lens cleaning device such as through force convection through airflow generated by the airflow device 10.

The foregoing description of preferred embodiments of the present disclosure has been presented for purposes of illustration and description. The described preferred embodiments are not intended to be exhaustive or to limit the scope of the disclosure to the precise form(s) disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the concepts revealed in the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. An airflow device for projecting a flow of air proximate to a field of view of a camera, the airflow device comprising:
a housing shaped to fit over an end of a camera lens;
one or more air pumps located within the housing, the one or more air pumps receiving air through one or more air inlets;
one or more air outlets formed in the housing proximate the camera lens, wherein the one or more air outlets are oriented to project air from the one or more air pumps across a field of view of the camera lens.

2. The airflow device of claim 1, the one or more air outlets comprising:
one or more projecting outlets oriented to project air from the one or more air pumps away from an end of the housing and substantially parallel to a field of view of the camera and
one or more lens outlets oriented to projected air from the one or more air pumps perpendicular to and across a field of view of the camera.

3. The airflow device of claim 1, the one or more air pumps comprising piezoelectric air pumps.

4. The airflow device of claim 3, wherein the one or more air pumps comprising piezoelectric air pumps are arranged in an array around the housing of the airflow device.

5. The airflow device of claim 4, wherein the piezoelectric air pumps are arranged in an array and are located within walls of the housing of the airflow device.

6. The airflow device of claim 4, the one or more air pumps further comprising at least one check valve in fluid communication with an interior of the one or more air pumps.

7. The airflow device of claim 1, the housing including a lens cover attached thereto and a camera chamber located between the lens cover and the camera when the airflow device is installed on the camera.

8. An airflow device for projecting a flow of air proximate to a field of view of a camera, the airflow device comprising:
a housing shaped to fit over an end of a camera lens;
one or more air pumps located within the housing, the one or more air pumps receiving air through one or more air inlets, the one or more air pumps comprising piezoelectric air pumps;
one or more air outlets formed in the housing proximate the camera lens, wherein the one or more air outlets are oriented to project air from the one or more air pumps across a field of view of the camera lens.

9. The airflow device of claim 8, wherein the one or more air pumps comprising piezoelectric air pumps are arranged in an array around the housing of the airflow device.

10. The airflow device of claim 8, the one or more air outlets comprising:
one or more projecting outlets oriented to project air from the one or more air pumps away from an end of the housing and substantially parallel to a field of view of the camera and
one or more lens outlets oriented to projected air from the one or more air pumps perpendicular to and across a field of view of the camera.

11. The airflow device of claim 8, wherein the piezoelectric air pumps are arranged in an array and are located within walls of the housing of the airflow device.

12. An airflow device for projecting a flow of air proximate to a field of view of a camera, the airflow device comprising:
a housing shaped to fit over an end of a camera lens;
one or more air pumps located within the housing, the one or more air pumps receiving air through one or more air inlets;
one or more air outlets formed in the housing proximate the camera lens, wherein the one or more air outlets are oriented to project air from the one or more air pumps across a field of view of the camera lens, the one or more air outlets comprising:
one or more projecting outlets oriented to project air from the one or more air pumps away from an end of the housing and substantially parallel to a field of view of the camera and
one or more lens outlets oriented to projected air from the one or more air pumps perpendicular to and across a field of view of the camera.
